(19) United States
(12) Patent Application Publication
(10) Pub. No.: US 2012/0288450 A1
(43) Pub. Date: Nov. 15, 2012

(54) Chewing gum formula for enhancing psycho-spirituality

(76) Inventor: Eduardo Jose Gonzales, Washington, DC (US)

(21) Appl. No.: 13/506,928

(22) Filed: May 25, 2012

Related U.S. Application Data
(63) Continuation-in-part of application No. 12/541,163, filed on Aug. 14, 2009, now abandoned.

(51) Int. Cl. A61K 9/68; A61P 25/00

(52) U.S. Cl. 424/48

(57) ABSTRACT

The present invention relates to a chewing gum formulation that serves as a means for awakening human consciousness and mindfulness to the sensorial subtleties, which in turn strengthens sovereignty such that overall psycho-spirituality is enhanced. More particularly, this invention relates to a dietary supplement consisting of the botanical plant Salvia divinorum as the source substance, including Salvinorin Alpha (A) as its primary active constituent, which is precisely extracted from S. divinorum to achieve a consistent dosing regimen predetermined for standardized efficacies.

### CROSS REFERENCE TO RELATED APPLICATIONS

100011 This application is a continuation in part of pending U.S. application Ser. No. 12/541,163, the disclosure of which is incorporated herein for all allowable purposes.

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

The present invention relates to the field of chewing gum formulations, more specifically to the use of Salvia divinorum, which has the compound Salvinorin A as its principle active moiety. The present invention relates further to the field of tools for enhancing human awareness and mindfulness in order to improve overall psycho-spirituality and to better enable personal success. “Psycho-spirituality” is defined as the study and practice of the mind's association with metaphysical, moral, and intrapersonal beliefs. It includes the totality of psychic processes, both as conceived by the general rationalistic outward viewpoint of the typical western scientific community, such as Freudian based, Behaviorism, Neuropharmacology, etc., and the more inward oriented spiritual viewpoint more typical of the religions of the east, such as Hinduism, Buddhism, Taoism, etc.

Salvia is one of three botanical genera commonly referred to as Sage and is the largest genus in the Lamiaceae (i.e. Mint) family. The other two genera that take the name “Sage” are Perovskia atriplicifolia (Russian Sage) and Phlomis fruticosa (Jerusalem Sage). The genus name Salvia derives its name from the Latin words ‘salveo’ and ‘salvare’, which mean ‘to heal’ and ‘to save’. The root takes meaning from the cultural context of the ancient Greeks using it to treat tuberculosis, ulcers, and snake bites. Similarly, the Romans would use Salvia for toothpaste and believed it to be good for the brain, senses, and memory. Since then, Sage has come to be known worldwide for its medicinal properties.

Salvia divinorum is an herbaceous species of Salvia and the commonly known culinary sage is Salvia officinalis. No species other than S. divinorum within this genus is known for inducing psychoactive effects, but Salvia splendens, which contains the neoclerodane diterpenoid compounds Salviarin and Splendidin, is considered by some to have a tranquilizing and sedative effect. Even the common, culinary sage has been reported as provoking a slight inebriation feeling if smelled for a prolonged time, due to it containing Thujone.

S. divinorum has an indigenous history in the western hemisphere, being that it is native to the Oaxaca region of Mexico where it is considered sacred and has been cultivated for centuries by the indigenous Mazatec shamans. In Mazatec culture, religion and medicine are far more intertwined than in Western culture, as evidenced by the Curanderos (“one who knows”), the specialized healers that administer the sacred plant in the form of an aqueous tea infusion of crushed leaves. S. divinorum is therefore traditionally used in religious ceremonies for spiritual healing, consciousness expansion, divination, and to enable visionary states of mind.

S. divinorum was first introduced into the western culture by Jean Johnson in 1939, but it wasn't properly cataloged until 1962 when Albert Hofmann and Gordon Wasson sent a botanical sample to Carl Epling and Carlos Játiva. A Mexican group led by Alfredo Ortega in 1982 isolated the active constituent, which would later be called “Salvinorin A”. The Leander Valdés team in 1984 also isolated the active constituent in a bioassay and presumed it to be the psychoactive constituent but it wasn't until Daniel Siebert performed the Heffter technique almost 10 years later that it was definitively proven as such (Ott 1995).

S. divinorum is known as “Diviner's Sage” but also Seer's Sage, Ska Maria Pastora, Hojas de la Pastora, Hierba de María, La Hembra, Mexican Mint, Magic Mint, Sally-D, Salvia, and a few other combinations of these. Although not suggestive by some of these pseudonyms, it is an herb with psychoactive properties that commonly induces dissociative effects. In low doses the five senses are enhanced and in moderate to high doses perception becomes extra-sensory. In the United States, neither S. divinorum nor any of its constituents, including Salvinorin A, are currently controlled under the federal Controlled Substances Act (DEA 2008). As of August 2009, eleven states have enacted legislation to control S. divinorum as a Schedule I drug. It is the opinion of this inventor after extensive study of the literature in the field that such legislation is not well founded and that the benefits of Salvinorin A far outweigh any of the alleged reasons given for enacting such restrictive legislation. In this application, the inventor provides his opinions as a result of his survey of the literature.

Salvinorin A's chemical makeup is C₂₃H₂₈O₈ and constitutes about 0.18% of a dried S. divinorum leaf (Ott 1995). It is specifically considered a trans-neoclerodane diterpenoid and thus belongs to an entirely different chemical class than any previously identified opioid receptor ligands, including other kappa-opioid receptor (KOR) agonists (Roth 2002, Prisinzano 2005). Salvinorin A is excreted via trichomes of the peltate-glandular morphology located just beneath the waxy cuticle layer (Siebert 2004, Kunkel 2004). While it is considered among the most potent naturally occurring psychoactive substances this inventor believes that it is almost entirely non-toxic based upon a survey of the toxicological literature he has performed and which in his opinion, has shown that Salvinorin A should be classified as non-toxic. The basis of his opinion includes the studies performed by Leander Valdés at the University of Michigan, Jeremy Stewart at the University of Mississippi, Frank Jaksch of Chromadex Inc., and Wayne Briner at the University of Kansas; Mowry et. al's findings (2003) also corroborate the low toxicity of Salvinorin A.

This inventor also believes that another salient characteristic of Salvinorin A is that it is non-habit forming and thus non-addictive, which minimizes the abuse potential inherent to alcohol, nicotine, and most other psychoactive drugs (Baggott 2004, DeHaven-Hudkins 2004). Unlike other opiates and even other KOR agonists, Salvinorin A does not induce the release of dopamine in the nucleus accumbens region of the brain that excites the brain reward system attributed to addictiveness (Grundmann 2007, Arias-Carrión 2007). Whereas Nicotine and Mescaline are alkaloids, Salvinorin A is a terpenoid and thus does not have a basic nitrogen atom even though it accepts oxygen atoms. Diterpenoids further classify terpenoids as a subset of them in having 4 isoprene units. Terpenoids are soluble in non-polar solvents like water and alcohol but only after freeing from their base compounds via the extraction process.

Salvinorin “B” is another innate constituent contained within S. divinorum, but it is not known to induce psychoactive effect. Salvinorin diterpenoids “D” an “E” have also shown no activity, but “C” and “F” are still inconclusive as such. Salvinorin “G” has also been isolated, along with Divinatorins “A” through “E” (Lee 2005). Other naturally occurring chemicals in Salvia divinorum are Loliolide, Hardwickiic acid, Methyl ester, Oleanolic acid, Presqualene alcohol, Peplusol, Stigmasterol, Neophytadiene, and 5-hydroxy-7-4′-dimethoxyflavone. There are also reports of other Flavonoids that have not been identified.

Chewing unextracted leaves is known. However, chewing unextracted leaves is very inefficient due to the limitations of saliva as a solvent and therefore upwards of 20 to 120 fresh or dried leaves are needed (Ott 1995) to achieve the same level of effect as when the present invention is used. Further, chewing unextracted leaves provides no discernible benefit to the user for 12 to 18 minutes, a subtle effect from 18 to 28 minutes, followed by a rapid increase in the amount delivered, providing a vivid or greater effect that can extend for an hour.

S. divinorum and its active constituents can also be smoked, vaporized, taken as an alcoholic (i.e. ethanol content greater than 40%) tincture, or in a quid.

The most common form of ingestion prior to the present invention was via the inhalation of smoke when the extract fortified leaves were burned. This form of ingestion is often accompanied by irritation of the lungs and coughing. The effects of smoking are felt within minutes, and typically provide a vivid psychoactive effect that lasts for less than 15 minutes, the effect dropping with time. When S. divinorum via Salvinorin A fortified leaves are ingested via smoke in large doses the effects are short lasting (i.e. between 14-17 minutes) but highly potent to the point that it is often hypnagogic and thus alarming (Siebert 2009). There is even a likelihood for dysphoric reactions (Carlezon 2006), where the probability is proportional to the dose. When abused as such and with an uninformed, inappropriate prior mindset, and/or in an inappropriate settings it is generally ill suited for psycho-spiritual or even recreational use. In addition, there is a physical danger of smoking in that there are risks of dropping the ignited burning contents of the paraphernalia while in the process of coming under the effects of the drug, which often occurs within just 45 seconds. Smoking also provides added dangers since the main by-products of pyrolysis (i.e. chemical decomposition of a condensed substance by heating) are mutagens and carcinogens. Furthermore there are inherent inefficiencies due to the high melting point (464° F.) of Salvinorin A.

In the field of medicament delivery, parenteral is most efficient for active agents but that intravenous technique is not practical due to S. divinorum's constituents being relatively insoluble in water, the costs of properly administering compounds in this manner, and the discomfort to the individual. Stability and shelf-life issues prevent nasal and oral sprays from being feasible; these techniques further suffer in cultural integration. Chewing the leaves provide for a more moderate effect but is not normally practical due to the bitter taste and unappealing texture.

### SUMMARY OF THE INVENTION

The present invention is a new method for delivering Salvia dDivinorum and/or active ingredients therefrom including Salvinorin A by ingestion/absorption from chewing gum. In particular, substantial delivery of Salvia divinorum and/or active ingredients therefrom including Salvinorin A are believes to be absorption through tissue in the mouth and throat. By mostly bypassing the gastrointestinal metabolism pathway, which breaks Salvinorin A down due to a monoamine oxidase function, this new method of delivering Salvinorin A, transfers the active ingredient from the gum base into the individual's nervous system buccally and sublingually via the mucous membranes in the mouth.

For use herein, the following scales are used, for drug concentration in micrograms of the active ingredient per kilogram body weight, the typical perceived psychoactive effect, and a term in the art for dosing which corresponds to this concentration:

| | | |
|---|---|---|
| 0.01-0.2 micrograms/kg body weight | Subtle effects | “1× dosing” |
| 0.20-0.8 micrograms/kg body weight | “Altered” effects | “5× dosing” |
| 0.8 to 1.4 micrograms/kg body weight | “Light” effects | “15× dosing” |

| | |
|---|---|
| 2+ micrograms/kg body weight | “Vivid” psychedelic |
| | effects, Smoking |

The gum of the present invention allows controlled release of the active ingredient, providing a low stable dose that is quickly achieved13 typically in 5 minutes or less—and are thereafter maintained constant. By low, one piece of the gum will provide sufficient Salvinorin A and at such a rate as to maintain for a period of between 15 and 60 minutes, typically between 20 and 30 minutes, a Salvinorin A content in the body of between 0.01 and 1.4 micrograms per kg for an average person. In preferred embodiments, one piece of the gum will provide sufficient Salvinorin A and at such a rate as to maintain for said between 15 and 60 minutes, typically between 20 and 30 minutes period a Salvinorin A content in the body of between 0.05 and 1.4 micrograms per kg for an average person, for example between 0.1 and 0.8 micrograms per kg for an average person, or for example about 0.2 to 0.5 micrograms per kg for an average person over the defined period of time.

By stable amount we mean that over a predetermined period of time, with normal chewing, the gum should establish and maintain said concentrations of active ingredient within the body, thereby providing a stable psychoactive effect, for said period of time. It is expected that with extended chewing the release rate may decline below replacement levels after some period of time, but in preferred embodiments the concentration of active ingredient in the user is maintained at within 50%, typically within 20%, of the “onset concentration” that is experienced within five or six minutes after the start of chewing, for at least 20 minutes, preferably for at least 30 minutes, and in some embodiments for at least 40 or 60 minutes.

### BRIEF DESCRIPTION OF FIGURES

FIG. 1 is a graph expressing on the y axis the predicted concentration of active ingredient retained in the user from both prior art methods of use (smoking and chewing) versus the concentration of active ingredients provided and maintained by a user chewing the gum of this invention.

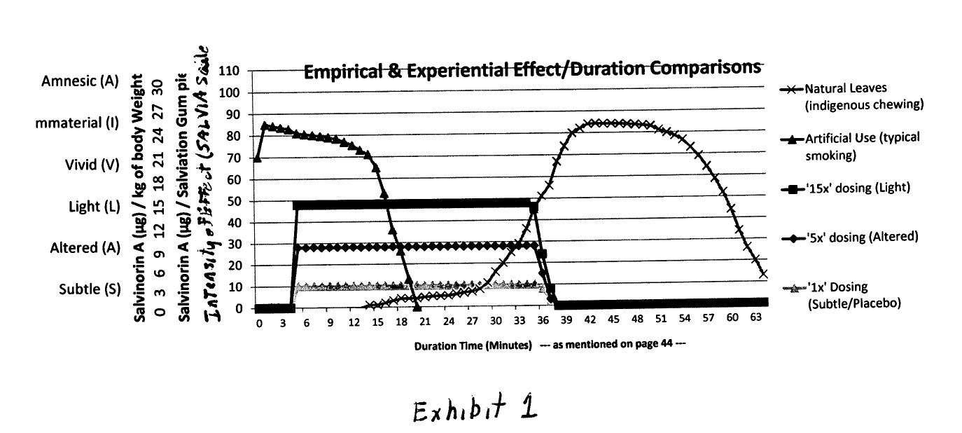

### DETAILED DESCRIPTION

The psychoactive effects, e.g., quick effect, of ingestion/absorption by mastication are similar to smoking, though chewing is superior because the duration of the effect produced is longer, its intensity is far more subtle, and the time to experience the first effect is greater in that it is at least five minutes. By delivering an amount sufficient to provide a low but stable level of active ingredients in the body, it is possible to enjoy moderate use of the material without unnecessary detrimental behavior.

Together, these improvements on the prior delivery methods dramatically lower the probability of dysphoria and give the individual ample time to gauge the effects and prepare accordingly. The progression of effects when chewing is as follows: (1) the effect begins to occur immediately after 5 minutes of mastication; (2) remains at this heightened sensation without diminishing for 30 minutes; and (3) the effects subside immediately and entirely, i.e. no more than 2-3 minutes after these 30 minutes; a feat that is confirmable using EEG scans of users' brains. Please note that this is similar to the effects of moderate and low dose ingestion of alcohol, but more importantly that this designed artwork (i.e. effect) has a duration and peak intensity profile that is entirely distinct from nature (i.e. artificially designed) and from any previous artwork in any field or craft. A problem with prior art methods is the intake of active ingredient does not match the elimination of the active ingredient by the body. As a result there tends to be periods of time when either smoking or chewing leaves or drinking tea where the concentration of the drug, and its effects, spike. And users can not readily maintain a controlled low effect. HPLC tests have shown that from 1 to 4 effective doses can be found in a single leaf where 0.2 mg is considered a minimal dose (Gruber 1999, Siebert 1994). The chewing gum formulation disclosed in the present invention is the most efficient and effective delivery technique, while also being among the least cumbersome and the most pleasant for the participating individual.

The psychoactive effect of Salvinorin A depends not only upon the method of ingestion but also on one's unique body chemistry. When inhaled while smoking high-doses the effect tends to dramatically impair one's motor skills, like alcohol, but potentially with the added danger of perceptual distortion. The present invention therefore combines Salvinorin A into a chewing gum formulation in low to moderate doses to minimize these inherent dangers and to bring the active ingredient's effect more closely into the realm that is commonly experienced from tobacco, alcohol, anesthetics, caffeine, methylphenidate, and many other common psychoactive compounds, including prescription drugs.

Salvinorin A has not been shown to incur any significant, competitive inhibition of reference target compounds at various different bioreceptor sites that are commonly effected by most other psychoactive compounds (Zhang 2005, Roth 2002, Chavkin 2004); it is therefore not considered analogous to the active ingredients in these other substances. This is further illustrated by the fact that it does not inhibit the effect of the Monoamine Oxidase Type-A (MAO-A) or Type-B (MAO-B) enzyme nor has it shown any active binding to the anandamide (CB1) or MK-801 receptor sites (Mechoulam 1998, Callaway 1998). As of yet this inventor is not aware of any known medically supported adverse health risks associated with S. divinorum use (Butler 2004), which is a great advantage over typical prescription and recreational drugs. Nor are there any known risks of overdose despite over 2 million Americans having tried it (SAMHSA 2008). And of course, one object of the present invention is to provide the active ingredient in a form where overdosing is difficult or substantially impossible, by putting the active ingredient in low amounts into a delivery vessel which releases the active ingredient over a long period of time. Also, there are currently no known occurrences of a fatal overdose or damage to organs.

Salvinorin A is not unanimously considered hallucinogenic, even at high doses. Instead, the proper, nuanced descriptors for the high dose effects of Salvinorin A are “oneirogenic” (Toro 2007) and “phantasticant” (Lewin 1924) since it induces hypnagogia (i.e. the transition to a delta-wave, dream-like state of consciousness that is otherwise known as lucid day-dreaming). Roth et al. (2004) further euphemizes the hallucinatory stigma by suggesting that the effects instead induce the perception of “spatio-temporal dislocation”. Indigenously, the descriptors are practically non-sequitur since the effects produce a state of inebriation that the Mazatecans would use for instruction, guidance, and for reaching a state of alleged divinity.

Salvinorin A in these moderate to low doses enhances psycho-spirituality by helping one engage in practical, self-intuitive questioning & answering while also inducing a greater self-confidence, centering, and a greater understanding of the world (particularly one's place in it). It produces a decreased sense of anxiety, fear, and doubt. Instead it enables an increased connection with the present moment and facilitates a sense of peace, insight, mood, comfort, connection with nature, and a feeling of calmness (Baggott 2004). Although the effects are mostly subjective, the effect does alter behavior and perception. Usage is therefore recommended only for mature and introspective individuals. The recommended environment is a quiet, controlled environment indoors, among tranquil music, and/or in a non-urban outdoor setting. Supervision with an informed or experienced sitter for first time users is highly recommended when taken in moderate or high doses but is not considered by this inventor as being so necessary for low doses. Other recommendations are to create a safe space and to plan one's time accordingly since the effect can last two to three hours in moderate and high doses. It is also highly recommended not to ingest with other medications without prior consultation with a Medical Doctor. When chewing the gum, the individual is recommended to expel the gum from the oral cavity if the experience becomes undesirable.

By following all the aforementioned recommendations in conjunction with the method of delivery presented herein, this inventor believes that the likelihood of a desirable effect is practically guaranteed for most individuals. In line with the psycho-spiritual effects, the use of S. divinorum will enhance one's existing practice (Crow) of eastern-originated forms of spirituality such as yoga and meditation (Soutar 2000, Ball 2007, Hanes 2003). It can also enhance one's personal philosophic practice as well, a field of practice dating back to ancient Greece.

The counter cultural elements and non-mainstream approaches of this invention are fully acknowledged regarding the uses and benefits of this invention. In the recently accelerated integration of new-age culture in the past few decades, there has been an increased interest in the use of botanicals and the themes of unity, harmony, and reconciliation of dualities. The present invention is uniquely effective in assisting individuals who wish to pursue the goal of attaining a conscious unity of spiritual and physical realities in the present moment (Arthur 2008). Such a pursuit via utilization of the present invention is entirely within the currently accepted righteous and moral intent inherent in religious and spiritual practice.

The need for spiritual improvement in our society without the often concomitant accompanying disadvantages of existing sense altering compounds is important due to the inherent power behind perceptual enhancements. Perception reaches climax when sensorial appreciation and attention is amplified in the present moment. When focused in the present moment, as the use of the present invention will facilitate, persistent dwelling on the past and/or anxiety of the future is minimized. As a result, moral decisions can be consciously made and sovereignty achieved when the crux of each matter at hand can be given full attention via unfettered thought processing in a realm of enhanced mindfulness. Correspondingly, collaborative business enterprise, ethical trade, and functional political interchange can be markedly enhanced with or without cognizance of the underlying improvements in personal psycho-spirituality, including breakthroughs reached in prior, private meditation.

Admittedly, psycho-spirituality can be enhanced via active or passive practice without using sense altering compounds. Active practice is exhibited via loving acts to others such as generosity, compassion, truth, unconditional love, forgiveness, sacrifice, justice, grace, and/or mercy. Passive practice can be performed via worship, honor, and glorification of a deity or deities as a person perceives them individually, and/or through an organized religion. The present invention better facilitates both of these ideals by introducing individuals to previously unaware of opportunities with an altered mindset. The concept of an altered mindset has historically in the west been denigrated due to a rational framework based exclusively on causality and circumscribed thought. However, anomalies such as dreams and the psychoactive effects in adults from nicotine, alcohol, prescription drugs, and caffeine are commonly accepted in spite of the disadvantages caused by the ingestion of some of these commonly used compounds.

In this inventor's opinion, Salvia divinorum is societally being unfairly characterized exclusively by its psychotomimetic effects when inhaled in high doses (i.e. above 1 mg of Salvinorin A). As a result, care should be taken to differentiate the varied effects from other psychoactives and separately evaluate the results of ingestion of Salvinorin A when at moderate and low doses (Siebert 1994). In this inventor's opinion, based upon his survey of the literature, future research in the use of Salvinorin A could lead to clinical improvements in a number of human health areas as a result of ingesting this botanical plant, such as improvements with depression in the Hanes' 2001 Case Report. Braida et al.'s 2009 anxiolytic and anti-depressant findings when performing in-vivo tests in mice corroborate this report and further broaden this compound's possibilities. An interesting finding of current research is the fact that the systemic delivery of S. divinorum does not increase serotonin levels, which is a trait common to some anti-depressants, and further that it decreases the caudate putamen dopamine levels. Future tests that consider and incorporate all of the botanical's diterpenes (i.e. not just Salvinorin A) is likely to reveal the mechanism behind these moieties' pharmacological nature in humans and further open the door to other mental and physical health benefits. In this inventor's opinion, future discouragement of scientific research in the use of Salvia divinorum, as in some states, would be a great loss to a humanity desperately in need of increased psycho-spirituality.

Thus far, various scientists from different research institutions have hypothesized that the active constituents of Salvia divinorum have a great potential for possible medicinal benefits. For instance, studies have concluded that the effect of Salvinorin A on reinstatement of extinguished amphetamine self-administration behavior is successful in decreasing the effect of cocaine-produced drug seeking and thus have demonstrated preliminary successes in treating addictions (Schenk 2001, Rothman 2000, Tidgewell 2004). This research is corroborated by another study by Thomas Prisinzano, who found that a rat would stop taking cocaine when given free access to both cocaine and Salvinorin A (Masis 2007).

In this inventor's opinion, there is also a potential for the use of S. divinorum as an analgesic and as a short-acting, general anesthetic. Existing narcotic analgesics (i.e. prescription pain-killers) consist of opioid receptors, including the kappa variety (McCurdy 2006, Trentini 2006, Wang 2005). S. divinorum exhibits antinociceptive properties by blocking both the physical suffering and perception of pain. It does so in a safer way, comparatively, by not depressing respiration or enabling the addiction tendencies associated with some other anesthetics or analgesic stimulants (Crow).

In other fields, studies have shown S. divinorum can inhibit motility common to diarrhea (Capasso 2008). Using S. divinorum to treat other digestive problems such as constipation, though, is only anecdotally verified by Mazatecan use. Similarly, little to no scientific evidence has surfaced to support the indigenous use of S. divinorum to treat anemia (Valdés 1983). Although research has suggested that this botanical can be used for sedative purposes (Butelman 2008, Fantegrossi 2005) there is no conclusive evidence as of yet for treating insomnia or stress. However in this inventor's opinion, these racing-brain pathologies would likely subside when a user becomes more attune to the present moment. Testimonials of S. divinorum use further corroborate the potential treatment in these fields.

The scientific evidence favoring using S. divinorum to treat arthritis and other forms of rheumatism (Siebert 2002), which is the colloquial term for issues with joints and tissue, is a little more promising when one examines the use of other kappa-opioids to treat inflammation (Walker 2001) and the anti-inflammatory evidence from Capasso's research of the gastrointestinal system. Similarly, other kappa-opioids have been shown to dilate arteries (Pei 2003), which in this inventor's opinion suggests possible treatment of congestive heart failure with the use of S. divinorum. Some of the other hypothesized potential health and therapeutic benefits of S. divinorum include the treatment of AIDS/HIV and Cancer (Chao 1998, Moran 2007). Cutaneously, there is also in this inventor's opinion, the potential for S. divinorum to act as a poultice by using a liquid application of the leaf on injured body parts (Ott 1995). Furthermore, this inventor believes there exists the potential treatment of other ailments such as sore throats, cold-symptomatic coughing, and headaches by the use of S. divinorum. This inventor also believes that S. divinorum may be useful as a diuretic to elevate the rate of urination and thus provide a means to treat drug overdose or poisoning from hemorrhagic cystitis.

When considering either the known health benefits or potential health benefits of Salvia divinorum it is pertinent to also reflect on the combined benefit when ingesting as part of a non-cariogenic chewing gum formulation since chewing gum can by itself provide various oral health benefits. For instance, chewing gum after meals helps stimulate the production of saliva for overall salivary flow which serves to wash away and neutralize the acid produced by bacteria in plaque (due to the hydrogen carbonate ion constituents), such plaque being responsible for dental decay, bad breath, and cavities. Chewing gum also helps relieve pressure in one's ears and sinuses through the repetitive jaw movement. Also, it can repair early tooth decay and strengthen tooth enamel due to the innate minerals in saliva (e.g. calcium, phosphate and fluoride) being components of tooth enamel that can be assimilated (Burt 2008). A study performed by the Baylor College of Medicine indicated that chewing gum can even lead to better academic performance. On another note, since two sticks of the gum in this invention are roughly 20 calories it can also help control weight gain as a low calorie supplement. Corroborating this statement, a study performed by Louisiana State University reported decreased craving for sweet foods and overall less hunger when chewing gum (Gajilan 2009). With health care and obesity being a national problem, anything that can help these problems should be welcome.

Obesity is one of among many health complications that burdens western health-care systems. In the continued search for novel approaches, recent research has suggested that many physical and mental health issues troubling our global societies can be prevented behaviorally. Scientists and general practitioners are therefore now targeting mood, disposition, and even spiritual practice (Seybold 2002) as potential sources for new forms of health care. That potential is reflected by the second most commonly reported subjective effect of taking Salvia divinorum, i.e. increased mood (Baggott 2004). Some users have more cogently reported learning transformative lessons when under the influence of this introspective-enhancing entheogen.

In the field of psychotherapy one potential use of S. divinorum's effects is induction of a profound state of self-reflection akin to hypnosis in order to better retrieve repressed childhood memories as a result of providing access to areas of the psyche that are ordinarily difficult to reach. Therefore this inventor believes that with just a few experiences with Salvinorin A, profound improvements can be made as opposed to the continuous medication regimen from prescription drugs that conventionally only offer symptomatic relief. The psychoactive Salvinorin A with its unique chemical properties therefore opens up a new pharmacological realm of future drug development (Siebert 2002). For example, S. divinorum may be useful for treating perceptual distortions and therefore a useful psychotherapeutic weapon against schizophrenia, dementia, and bipolar disorder (Roth 2002). Other potential treatments could be for Alzheimer's (Roth 2002, Barg 1993, Mathieu-Kia 2001).

Although the proof of effectiveness of Salvinorin A in the medicinal and scientific supplement arena is either promising or ongoing, in many fields it is already well documented. While the viable use of S. divinorum as an anti-depressant is still inconclusive, its research is one of the fields of study most pertinent to this invention. The byproduct of these disorders are behavioral in nature and thus, arguably, controllable from a psycho-spiritual perspective within one's own mental faculties. This inventor believes that the use of this invention will ultimately demonstrate that true mental suffering is tied to one's associated ego and identity, which is derived from one's remembered past, anticipated future events, and often accompanied by persistent dwelling on these events, with resulting chronic anxiety. The anti-depressant effects of S. divinorum, studied by this inventor in review of the literature, may therefore not be chemical but instead psycho-logical and/or psycho-spiritual. Coupling this with the growing trend in using non-stimulants (Michelson 2001) and anti-depressants to treat attention deficit disorders and hyperactivity disorders (Higgins 1999, Caron 1999) substantiates this inventor's premise that using S. divinorum can enhance one's present-moment attention and spiritual intention on personal growth.

The need for a qualitatively high level of spirituality, including with atheists, has always been dire in functional societies and inter-societal relationships. With continually increased global, socio-economic networks over the past several decades the opportunity to enrich communication and encourage cooperation amongst developed and developing countries has never been so great. Traditional methods of interaction have repeatedly failed as a result of circumscribed and exclusionary tactics, as proven by the numerous wars over the past 150 years, disparities in economic comfort, and general malcontent often expressed even in the most fortunate of individuals or countries. A paradigm shift is therefore necessary, which will give credence to new or newer techniques of increasing general psycho-spiritual wellness. To date there has not been a method of increasing psycho-spirituality that would be amenable for busy persons who did not want to “drop out” of the current western culture. This method is an alternative to the time consuming methods now being employed with their various numerous disadvantages.

There are many patents related to chewing gum, some that are not exclusively for confectionery purposes, and a few containing plants and compounds from the Sage genus. However, none of them address the novel, non-obvious combination of purpose and function of the invention described herein, because they do not address, contemplate, nor are they specifically designed for the purpose of enhancing psycho-spiritual abilities and personal success.

Examples of various uses of chewing gum include:

U.S. PAt. No. 7,422,760 granted Sep. 9, 2008 to S. Zhong et al. (Plant-Based Medicament for the Treatment of Hepatitis C). U.S. PAt. No. 7,078,052 granted on Jul. 18, 2006 to R. L. Ream et al. (Pharmaceutical chewing gum formulations). Only claims the use of caffeine. U.S. Pat. No. 7,081,211 granted on Jul. 25, 2006 to Y-j Li et al. (Multi-layer reaction mixtures and apparatuses for delivering a volatile component via a controlled exothermic reaction). U.S. Pat. No. 6,852,345 granted on Feb. 8, 2005 to V. A. Hill et al. (Stabilized chewing gum base). Does not claim the addition of a medicament or an active ingredient. U.S. Pat. No. 6,787,167 granted on Sep. 7, 2004 to B-J Stahl (Use of natural vegetable components as flavoring agents in chewing gum coatings). U.S. Pat. No. 6,664,225 granted on Dec. 16, 2003 to J. A. Mumoli (Single-dose quick-dissolving cleansing agent with medicinal properties). Use of Salvia officinalis (a different species) for treating bipolar disorder or schizophrenia.

U.S. Pat. No. 6,380,175 granted on Apr. 30, 2002 to A. A. Hussain et al. (Method for enhancement of delivery of THC by the administration of its prodrugs via the nasal route). U.S. 6,358,060 granted on Mar. 19, 2002 to J. M. Pinney et al. (Two-stage transmucosal medicine delivery system for satisfying a nicotine craving). U.S. Pat. No. 6,328,992 granted on Dec. 11, 2001 to L. L. Brooke et al. (Cannabinoid patch and method for cannabis transdermal delivery). U.S. 6,132,762 granted on Oct. 17, 2000 to W. Cristobal (Transcutaneous application of marijuana).

U.S. Pat. No. 6,248,760 granted on Jun. 19, 2001 to P. Wilhelmsen (Tablet giving rapid release of nicotine for transmucosal administration). U.S. Pat. No. 5,866,179 granted on Feb. 2, 1999 to E. S. Testa (Medicated chewing gum and a process for preparation thereof). Presents a medicated gum for satisfying a nicotine craving. Does not mention the use of Salvia divinorum or Salvinorin A. Focuses on the formulation and manufacturing techniques for heat sensitive agents, but Salvinorin A is not known to be heat sensitive under 464° F. U.S. Pat. No. 5,593,684 granted on Jan. 14, 1997 to R. W. Baker et al. (Method and therapeutic system for smoking cessation). U.S. Pat. No. 5,549,906 granted on Aug. 27, 1996 to G. C. Santus (Nicotine lozenge and therapeutic method for smoking cessation). U.S. Pat. No. 5,512,306 granted on Apr. 30, 1996 to T. Carlsson et al. (Smoking substitute).

U.S. Pat. No. 5,488,962 granted on Feb. 6, 1996 to G. Perfetti (Chewing gum as a substitute for tobacco smoke). U.S. Pat. No. 5,055,478 granted on Oct. 8, 1991 to T. M. Cooper et al. (Method for stopping smoking). U.S. Pat. No. 3,901,248 granted on Aug. 26, 1975 to S. Lichtneckert et al. (Chewable smoking substitute composition). U.S. Pat. No. 3,877,468 granted on Apr. 15, 1975 to S. Lichtneckert et al. (Chewable tobacco substitute composition). U.S. Pat. No. 2,536,168 granted on Jan. 2, 1951 to T. C. Goggin et al. (Amphetamine Chewing Gum). U.S. Pat. No. 2,262,087 granted on Nov. 11, 1941 to K. A. Bartett (Chewing Gum Tablet). U.S. Pat. No. 865,026 granted on Sep. 3, 1907 C. Ellis (Masticable Tobacco Preparation). U.S. Patent App No. 20090087501 published on Apr. 2, 2009 by D. Cummins (Oral Compositions Containing Botanical Extracts). Presents an oral composition for systemic health and oral-health (e.g. anti-microbial, anti-tartar), breath freshening, and malodour control. U.S. Patent App No. 20080233220 filed on Sep. 25, 2008 by S. Zhong et. al. (Further Medical Use Of A Botanical Drug Or Dietary Supplement). Botanical drug to treat various medical complications other than Hepatitis (e.g. Liver inflammation).

U.S. Patent App No. 20080020050 filed on Jan. 24, 2008 by T. L. Chau (Medicinal delivery system, and related methods). Presents a medicated gum for satisfying a nicotine craving. U.S. Patent App No. 20070190187 filed on Aug. 16, 2007 by B. W. Kneller et al. (Formulation for enhanced delivery of phenethylamine). Claimed use is within a dietary supplement as an alkaloid and a MAO-B enzyme inhibitor. U.S. Patent App No. 20070248693 filed on Oct. 25, 2007 by E. Mazzio et al. (Nutraceutical composition and method of use for treatment/prevention of cancer). U.S. Patent App No. 20070134299 filed on Jun. 14, 2007 by B. C. Giles (Method and formula for prevention of cold and flu and suppression of related symptoms).

U.S. Patent App No. 20070014887 filed on Jan. 18, 2007 by S. R. Cherukuri (Medicated chewing gum delivery system for nicotine). U.S. Patent App No. 20060073189 filed on Apr. 6, 2006 by J. M. Pinney (Chewing gums, lozenges, candies, tablets, liquids, and sprays for efficient delivery of medications and dietary supplements). U.S. Patent App No. 20040191334 filed on Sep. 30, 2004 by P-C Shaw et al. (Use of transhinone derivates as cholinesterase inhibitors in treating related diseases). Use of sublingual tablets via the use of the root of Salvia divinorum but primarily via the use of the root extract from Salvia miltiorrhiza (a different species). U.S. Patent App No. 20040180007 filed on Sep. 16, 2004 by R. L. Ream et al. (Pharmaceutical chewing gum formulations). General gum formulation with a medicament or agent such as caffeine. U.S. Patent App No. 20030050334 filed on Mar. 13, 2003 by R. B. Murty et al. (Process for extraction of Delta-9-Tetrahydrocannabinol and other related cannabinoids and preparation of specific strength marijuana cigarettes).

Publications include

Zhang, Y.; Butelman, E. R.; Schlussman, S. D.; Ho, A.; Kreek, M. J. (May 2005). “Effects of the Plant-Derived Hallucinogen Salvinorin A on Basal Dopamine Levels in the Caudate Putamen and in a Conditioned Place Aversion Assay in Mice: Agonist Actions at Kappa-Opioid Receptors”, Psychopharmacology Vol. 179 (3): 551-558.

Mowry, M.; Mosher, M.; Briner, W. (July 2003). “Acute Physiologic and Chronic Histologic Changes in Rats and Mice Exposed to the Unique Hallucinogen Salvinorin A” (PDF), Journal of Psychoactive Drugs 35 (3): 379-382, PMID 14621136

Siebert, D. J. (June 1994). “Salvia divinorum and Salvinorin A: New Pharmacologic Findings”, Journal of Ethnopharmacology 43 (1): 53-56, doi:10.1016/0378-8741(94)90116-3, PMID 7526076.

Siebert, D. J. (2002). “A Prominent Salvia divinorum Researcher Speaks Out: Letter to Congress (RE: Bill H.R. 5607)”. The Entheogens and Drug Policy Project. Center for Cognitive Liberty & Ethics.

Siebert, D. J. (2004). “Localization of Salvinorin A and Related Compounds in Glandular Trichomes of the Psychoactive Sage, Salvia divinorum”. Annals of Botany (Oxford University Press) 93 (6): 763-71. doi:10.1093/aob/mch089. PMID 15087301

Siebert, D. J. (June 2009). “The Salvia divinorum FAQ”. The Salvia divinorum Research and Information Center. http://sagewisdom.org/faq.html.

Siebert, D. J. (May 2009). “The Salvia divinorum User's Guide”. The Salvia divinorum Research and Information Center. http://www.sagewisdom.org/usersguide.html.

Baggott, M., Erowid, E. & F. (June 2004). “A Survey of Salvia divinorum Users”. Erowid Extracts Vol. 6: 12-14.

Hanes, K. R. (December 2001). “Antidepressant Effects of the Herb Salvia divinorum: A Case Report”, Journal of Clinical Psychopharmacology Vol. 21 (6): 634-635.

Ott, J. (1995). “Ethnopharmacognosy and Human Pharmacology of Salvia divinorum and Salvinorin A. Curare Vol. 18 (1): 103-29.

Prisinzano, T. E., Tidgewell, K., & Harding, W. W. (2005). “Kappa-Opioids as Potential Treatments for Stimulant Dependence”. AAPS J. Vol. 7 (3): E592-E599.

Roth, B. L., Baner, K., Westkaemper, R., Siebert, D., Rice, K. C., & Steinberg, S., et. al. (2002). “Salvinorin A: A Potent Naturally Occurring Non-nitrogenous Kappa Opioid Selective Agonist”. Proceedings of the National Academy of Sciences of the United States of America, Vol. 99 (18): 11934-11939.

Kunkel, D. (2004). “Leaf Glandular Trichome (Salvia divinorum)”. Scientific Stock Photography Library of Light Microscope Pictures and Electron Microscopy Images Featuring Science and Biomedical Microscopy Photos. Dennis Kunkel Microscopy, Inc.

Grundmann O., Phipps S. M., Zadezensky I., Butterweck V. (2007). “Salvia divinorum and Salvinorin A: An Update on Pharmacology Andanalytical Methodology”. Planta Med Vol. 73: 1039-1046.

Arias-Carrión, O., Poppel, E. (2007). “Dopamine, Learning and Reward-Seeking Behavior”. Act Neurobiol Exp Vol. 67 (4): 481-488.

Tidgewell, K.; Harding, W.; Holden, K.; Dersch, C.; Butelman, E.; Rothman, R.; Prisinzano, T. (September 2004). “Neoclerodane Diterpenes as Potential Drug Abuse Therapeutics”. The AAPS Journal 6 (Theme issue: Drug Addiction—From Basic Research to Therapies (AAPS-NIDA Symposium Frontiers in Science).

Rothman, R. B., Gorelick D. A., Heishman S. J. (April 2000). “An Open-Label Study of a Functional Opioid Kappa-Antagonist in the Treatment of Opioid Dependence”. Journal of Substance Abuse Treatment Vol. 18 (3): 277-281.

Lee, D. Y., Zhongze M., Liu-Chen L. Y., Wang, Y., Chen Y., Carlezon W. A., Jr. and Bruce Cohend, (March 2005). “New Neoclerodane Diterpenoids Isolated from the Leaves of Salvia Divinorum and their Binding Affinities for Human Kappa-Opioid Receptors”. Bioorganic & Medicinal Medicinal Chemistry Vol. 13: 5635-5639.

Carlezon, W. A.; Béguin, C.; DiNieri, J. A. (2006). “Depressive-Like Effects of the κ-Opioid Receptor Agonist Salvinorin A on Behavior and Neurochemistry in Rats”. Journal of Pharmacology and Experimental Therapeutics 316 (1): 440-447, doi:10.1124/jpet.105.092304, PMID 16223871.

Braida D., Capurro V., Zani A., Rubino T., Viganó D., Parolaro D., Sala M. (May 2009). “Potential Anxiolytic and Antidepressant-Like Effects of Salvinorin A, The Main Active Ingredient of Salvia divinorum, in Rodents”. Department of Pharmacology, Chemotherapy and Medical Toxicology, University of Milan, Milan, Italy.

Butler, L. (February 2004). “Awareness Training On Salvia divinorum”, Navy Personnel Command Drug Detection and Deterrence Branch (Pers-671).

SAMHSA (February 2008). “The NSDUH Report: Use of Specific Hallucinogens: 2006”. Substance Abuse and Mental Health Services Administration, Office of Applied Studies.

Gruber, J. W., Siebert D. J., Der Marderosian, A. H., Hook, R., (1999). “High Performance Liquid Chromatographic Quantification of Salvinorin A from Tissues of Salvia divinorum”. Phytochemical Analysis Journal.

Chavkin, C.; Sud, S.; Wenzhen, J.; Stewart, D. J.; Zjawiony, J. K.; Renock, S.; Baner, K.; White, N. M.; Pintar, J.; Roth, B. L. (2004). “Salvinorin A: An Active Component of the Hallucinogenic Sage Salvia divinorum is a Highly Efficacious Kappa-Opioid Receptor Agonist: Structural and Functional Considerations”. J. Pharm. Exp. Ther. Vol. 308: 1197-1203.

Toro, G., Thomas, B. (2007). “Drugs of the Dreaming; Oneirogens, Salvia divinorum and Other Dream-Enhancing Plants.” Park Street Press.

Lewin, L. (1924). “Phantastica: A Classic Survey on the Use and Abuse of Mind-Altering Plants”. Park Street Press.

Seybold, K. S., Hill, P. C. (Feb. 2002). “The Role of Religion and Spirituality in Mental and Physical Health”. Current Directions in Psychological Science. A Journal of the Association for Psychological Science.

Gajilan, A. C. (2009). “Chew on this: Gum May be Good for Body, Mind”. Special to CNN. http://www.cnn.com/2009/HEALTH/04/22/chewing.gum.benefits/index.html.

Burt, B. A. (2008). “Benefit of Chewing Gum on Teeth Health” Dental Health Magazine. http://worldental.org/gums/benefit-of-chewing-gum-on-teeth-health.

Caron, M., Gainetdinov, R. (January 1999). “Serotonin May Hold Key to Hyperactivity Disorder”. Howard Hughes Medical Institute (HHMI), Duke University. http://www.hhmi.org/news/caron2.html.

Michelson, D., Faries, D., Wernicke, J., Kelsey, D., Kendrick, K., Sallee, F. R., Spencer, T. (April 2001). “Atomoxetine in the Treatment of Children and Adolescents With Attention-Deficit/Hyperactivity Disorder: A Randomized, Placebo-Controlled, Dose-Response Study”. Pediatrics Journal Vol. 108 (5): 83.

Higgins, E. S. (January 1999). “A Comparative Analysis of Antidepressants and Stimulants for the Treatment of Adults with Attention-Deficit Hyperactivity Disorder”. Family Practice Journal 48 (1): 15-20.

McCurdy, C. R., Sufka, K. J., Smith, G. H., Warnick, J. E., Nieto, M. J. (January 2006). “Antinociceptive Profile of Salvinorin A, a Structurally Unique Kappa Opioid Receptor Agonist”. Journal on Pharmacology Biochemistry Behavior. Vol. 83 (1): 109-13.

Trentini, F. J., French, L. G., Erlichman, J. S. (September 2006) “The Antinociceptive Effect of Salvinorin A in Mice”. European Journal of Pharmacology Vol. 545 (2-3): 129-133.

Wang, Y., Tang, K., Inan, S., Siebert, D., Holzgrabe, U., Lee, D. Y. et al. (January 2005). “Comparison of Pharmacological Activities of Three Distinct Kappa Ligands (Salvinorin A, TRK-820 and 3FLB) on Kappa-Opioid Receptors in Vitro and Their Antipruritic and Antinociceptive Activities in Vivo”. Journal of Pharmacology and Experimental Therapeutics Vol. 312 (1): 220-230.

Capasso, R., Borrelli, F., Zjawiony, J., Kutrzeba, L., Aviello, G., Sarnelli, G. et al. (2008). “The Hallucinogenic Herb Salvia divinorum and its Active Ingredient Salvinorin A Reduce Inflammation-Induced Hypermotility in Mice.” Neurogastroenterology and Motility: the Official Journal of the European Gastrointestinal Motility Society Vol. 20 (2):142-148.

Walker, J. S. (October 2001). “Anti-Inflammatory Effects of Kappa-Opioids: Relevance to Rheumatoid Arthritis” Pain Reviews Vol. 8 (7): 113-119.

Valdés, L. J. III, Diaz, J. L., Paul, A. G. (May 1983). “Ethnopharmacology of ska Maria Pastora (Salvia divinorum, Epling and Jativa-M.)”. Journal of Ethnopharmacology Vol. 7 (3): 287-312.

Butelman, E. R.; Prisinzano, T. E., Deng, H., Rus, S., Kreek, M. J. (November 2008). “Unconditioned Behavioral Effects of the Powerful-Opioid Hallucinogen Salvinorin A in Nonhuman Primates: Fast Onset and Entry into Cerebrospinal Fluid”. Journal of Pharmacology And Experimental Therapeutics DOI: 10.1124.

Fantegrossi, W. E., Kugleb, K. M., Valdés, L. J. III, Koreedad, M., Woods, J. H. (August 2005) “Kappa-Opioid Receptor-Mediated Effects of the Plant-Derived Hallucinogen, Salvinorin A, on Inverted Screen Performance in the Mouse”. Behavioural Pharmacology Vol. 16: 627-633.

Roth, B. L., Feng, Y. (July 2004) “Salvinorin A: A novel and Highly Selective Kappa-Opioid Receptor Agonist”. Life Sciences Journal Vol. 75: 2615-2619.

DeHaven-Hudkins, D. L., Dolle, R. E., (2004). “Peripherally Restricted Opioid Agonists as Novel Analgesic Agents.” Current Pharmaceutical Design Vol. 10 (7): 743-757.

Crow, D. “Salvia divinorum, Diviners Sage: A Natural Anti-Depressive, Anti-Addictive Kappa-Opioid Agonist”. Holistic Consultant. httn://members.shaw.ca/duncancrow/salvia_kappa_opioid.html.

Schenk, S., Partridge, B. (April 2001). “Effect of the Kappa-Opioid Receptor Agonist, U69593, on Reinstatement of Extinguished Amphetamine Self-Administration Behavior”. Journal on Pharmacology Biochemistry Behavior. Vol. 68 (4): 629-634.

Soutar, I., Strassman, R. (January 2000). “Meditation with Salvia divinorum/Salvinorin A”. Multidisciplinary Association for Psychedelic Studies. http://www.maps.org/research/salvia/sdmeditation.html.

Ball, M. W (August 2007). “Sage Spirit: Salvia divinorum and the Entheogenic Experience”. Kyandara Publishing.

Hanes, K. R. (2003). “Salvia divinorum: Clinical and Research Potential”. Cognitive-Behavioural Treatment Centre. MAPS Vol. 13 (1).

Arthur, J. D. (March 2008). “Peopled Darkness: Perceptual Transformation through Salvia Divinorum”. IUniverse Publishing.

Pei, J. M., Chen, M., Wang, Y. M., Wen, J., Zhu, Y. L. (February 2003). “Kappa-Opioid Receptor Stimulation Contributes to Aortic Artery Dilation Through Activation of K(ATP) Channel in the Rats”. Sheng Li Xue Bao Vol. 55 (1): 91-95.

Barg J., Belcheva, M., Rowinski, J., Ho, A., Burke, W. J., Chung, H. D., Schmidt, C. A., Coscia, C. J. (1993). “Opioid Receptor Density Changes in Alzheimer Amygdala and Putamen.” Brain Research Vol. 632 (1-2): 209-215.

Mathieu-Kia A. M., Fan, L. Q., Kreek, M. J., Simon, E. J., Hiller, J. M. (2001). “Mu-, Delta- and Kappa-Opioid Receptor Populations are Differentially Altered in Distinct Areas of Postmortem Brains of Alzheimer's Disease Patients.” Brain Research Vol. 893 (1-2):121-134.

Masis, J. (February 2007). “Feature: Mexican drug gains U.S. following”. AlertNet (Reuters). http://www.alertnet.org/thenews/newsdesk/N24424552.htm.

Moran, T., Culhane, M. (October 2007). “Parents Blame Exotic Plant for Son's Suicide: Legal and Little Known Plant Packs Hallucinogenic Punch”. Nightline (ABC News). http://abcnews.go.com/Nightline/Story?id=3685391&page=2.

Chao, C. C., Gekker, G., Hu, S., Kravitz, F., Peterson, P. K. (1998). “Kappa-Opioid Potentiation of Tumor Necrosis Factor-Alpha-Induced Anti-HIV-1 Activity in Acutely Infected Human Brain Cell Cultures”. Biochemical Pharmacology Vol. 56 (3): 397-404.

Drug Enforcement Agency (November 2008). “Salvia divinorum and Salvinorin A”. Office of Diversion Control: Drug & Chemical Evaluation Section. http://www.usdoj.gov/dea/concern/salvia_divinorum_one_pager.pdf

The listing of these patents and publications is not an admission that any listed patent or publication is relevant prior art, but the disclosures of the references are incorporated herein by reference thereto for any allowed purpose.

A primary object of the invention is to provide a better means for improving the overall psycho-spirituality of participants by enhancing awareness and mindfulness. Another object of the invention is to enhance overall psycho-spirituality without requiring as much dedication, determination, and discipline as current methods of achieving spiritual enlightenment require. Another object of the invention is to enhance overall psycho-spirituality and thereby reduce the stresses and strains from daily modern life. A further object of the invention is to provide a method of enabling personal success while still staying engaged in an active western or other similar culture. Other objects and advantages of the present invention will become apparent from the following descriptions when taken in connection with the accompanying embodiment of the presently disclosed invention.

One object of the present method is not merely to disclose the chewing gum formulation but also its use and integration of the resulting experientially learned lessons, when chewing this gum, into a larger personal framework of self transformation. The beginning, supplementary, and/or essential support of such self transformation is presented in this invention via a natural and consciousness expanding confectionery. The inherent psycho-spiritual effect of chewing this Salvinorin A active is the realization that there is far more to the present moment than one is normally aware of.

Overall, this inventor believes, based upon his survey of the relevant literature, that the benefits of using the present invention over other activities such as alcohol, nicotine, caffeine, or prescription drug consumption are: (A) It is non-addictive; (B) There have been no reported withdrawal symptoms; (C) There have been no reported fatal overdoses; (D) It is likely to have medicinal benefits; (E) It is more effective in lower dosages; (F) It is natural; (G) It is likely to be less expensive; (H) It enables awareness expansion and effectively increases psycho-spirituality; and (I) It is practically non-toxic and therefore avoidant of many harmful side effects

Chewing Gum, for the purposes of this invention, is defined as any substance chewed in the oral cavity to provide medicinal or dietary supplements transmucosally to the user by mostly bypassing gastrointestinal metabolism. Transmucosal delivery implies buccal, sublingual, and pharyngeal absorption into the central nervous system. Absorption from chewing gum not only happens in the mucous membranes of the mouth but also marginal amounts are absorbed in the throat, esophagus, and larynx. Chewing provides for a synergistic effect where the sum total of the actives is greater than the individual parts.

One embodiment of the present invention provides a method for the chewing gum delivery of Salvia divinorum with a slightly saliva-soluble powdered blend of Salvinorin A and non-cariogenic sweeteners. The incorporation of sweeteners is imperative for realistic function as a viable invention to improve on the negative organoleptic (i.e. taste, texture, color, and odor) qualities associated with the ingestion of natural botanicals such as S. divinorum. In the preferred embodiment, the extracted active constituents of S. divinorum are included as part of the chewing gum formulation in three different doses. The chewing gum formulation and process, as discussed herein, is tailored specifically for the active compound Salvinorin A.

Some of the novelty presented herein originates from the fact that Salvinorin A doesn't efficiently enter the blood stream when ingested orally, due to the delays in its crossing the epithelial membranes and destruction of the active ingredient in the stomach. These delays account for the deactivation of the moiety when undergoing first pass metabolism and due to competing gastrointestinal reactions, both of which are common issues encountered by most orally delivered pharmaceuticals. Furthermore, when chewing un-extracted leaves, the saliva secreted from the mouth is not a strong enough solvent to extract Salvinorin A fast enough from its embedment within the plant's cuticle such that it can be absorbed in the bloodstream so as to reach the brain at pharmacologically effective levels. Incorporating pre-extracted Salvinorin A into the chewing gum formulation provides a more optimal effectiveness and efficiency.

This method for enhancing psycho-spirituality is basically comprised of the steps to prepare a chewing gum formulation, consisting of Salvia divinorum and its extracted diterpenes, and then ingesting the formulation by chewing the gum and swallowing the released ingredients. The chewing gum formulation can be selected in the manufacturing process so that it releases a sufficient dose for the subsequent efficient absorption of Salvinorin A while providing an organoleptically acceptable experience. The prime active constituent, Salvinorin A, can be extracted to powder form by employing an efficient and effective solvent means. The manufacturing process presented herein also provides for the Salvinorin A stabilization and minimal degradation.

The Salvinorin A powder can be combined with a non-cariogenic sweetener. The powder can also be measured and weighed into precisely measured doses to create a standardized dosing regimen relative to a standard level-of-effect scale. The time release of the active and non-cariogenic sweetener may also be balanced such that an individual can subjectively gauge and be able to predict further effects from the experienced intensity of the sweetness. There are a number of mechanisms to control release rate by gums, including incorporation of certain ingredients to retard release of the active ingredient, including for example hydrophobic materials, surfactants, emulsifiers, and other materials known in the art for controlling release rate of material from gum being chewed.

When still embedded in the leaf, the active component of S. divinorum is water- and saliva-insoluble so a base emulsifier system has added importance in this invention to improve the lipophilic balance of the active ingredients when incorporated together with the gum base. Base Emulsifier Systems also help to emulsify liquids, absorb moisture, and facilitate release of the active ingredients upon mastication. Emulsifiers generally balance out the consistency in a liquid suspension of any incompatible constituents of the formulation presented herein before or after commencement of mastication. The buffering agents are added to set the pH to within +/−2.5 of optimal and efficient absorption of Salvinorin A.

Alternative delivery means can also be created by embedding Salvia divinorum and/or its pre-extracted Salvinorin A constituent into hard confectionery such as lozenges, orally dissolved tablets, quids, disintegrating sweetened leaves, flavored granulation compounds for use in shishas or hookahs, sweet-tea infusions, and toothpaste. A disintegrating sweetened leaf, which is essentially a flavor-enhanced extract-fortified leaf, is another embodiment of this invention. In this means, minimal chewing in the oral cavity would be required to take place due to the compound dissolving and disintegrating completely in the mouth. This would give a similarly quick release and absorption pattern of Salvinorin A as presented in the chewing gum formulation. Another embodiment would be to use as a delivery vehicle the simple dipping of a Salvia divinorum leaf into a sweet-Salvinorin A solution, then rolling one or more leaves together into a cylindrical shape that would be consumed directly in the rolled leaf form. While not all of these delivery methods can facilitate high levels of sublingual absorption, any lack of efficiency can be addressed by a dosage increase calibrated for the desired probability of absorption into the blood stream to ensure that these preferred embodiments are also consistent with the three-level dosage approach.

It may be desirable to provide a fast release portion of the active ingredient disposed on or in the gum, and a slow release portion of the active ingredient in the gum, thereby achieving the desired body concentration of active ingredient in a short time, for example less than 5 minutes, while providing a portion of the active ingredient at a slower rate to provide a balance against the bodies destruction of the active ingredient once the desired body concentration is reached.

Alternative methods for enhancing psycho-spirituality can also use chewing gum formulations comprised of botanicals or botanical extracts such as Mitragyna speciosa, Rivea corymbosa, Ipomoea violacea, Cannabis sativa, Lophophora williamsii, Sceletium tortuosum, Banisteriopsis caapi, Psychotria viridis, Argyreia nervosa, Peganum harmala, Echinopsis pachanoi, Piper methysticum, Datura inoxia, Datura stramonium, Atropa belladona, Psilocybe cubensis, Phalaris arundinacea, Achnatherum robustum, Paullinia cupana, Artemisia absinthium, Acorns calamus, Heimia salicifolia, Calea zacatechichi, Amanita Muscaria, and Tabernanthe iboga, or combinations thereof. These single formulations can also be combined into a mixture that is then ingested in a chewing gum form.

In addition to Salvinorin A, unextracted leaf fragments from the botanical Salvia divinorum can be added to the chewing gum formulation presented in this invention. This formulation would include a gum base composition with one or more other excipients such as hydrophilic sweeteners, mostly insoluble fillers (e.g. elastomers, resins, fats, waxes, and oils), elastomer plasticizers, emulsifiers, diluents, softeners, water insoluble flavoring agents, water soluble buffer chemicals, antioxidants, humectants, abrasives, binders, disintegrants, surfactants, anti-adherents, encapsulating coating, glidants, lubricants, preservatives, sorbents, whiteners, and fluoride. This embodiment can also be used together with gum containing extracted Salvinorin A, to provide both the desired level and also a more “natural” experience.

The delivery system presented herein provides a consistent qualitative and quantitative ratio of the Salvinorin A active with the non-active compounds to achieve a reliable release rate, discussed further herein. For the sake of consistency and product efficiency this invention only provides the precise amount of Salvinorin A that is characterized for inducing a Pharmacological Effect (LPE) ranging from a minimal level to two higher levels of activity.

The primary goal of this invention is to help facilitate or enhance the mental states of one or more of the following: psycho-spirituality, existential religious fervor, meditation, ontological insight, perception, psychological disposition, psychotropic activity, present-moment attention, consciousness expansion, righteousness development, mysticism enabling, faith strengthening, spiritual revelation, and ego-dissociation.

A secondary goal of this invention is the potential research benefits in the areas of medicine, health, and pharmaceuticals which will result from this invention's being able to allow the easily dosed and measured ingestion of S. divinorum and/or its active constituents.

An embodiment of the invention provided as part of the chewing gum formulation is the incorporation of both unextracted Salvia divinorum leaf specks and the extracted Salvinorin A diterpene in order to provide a balanced product while also ensuring pharmacologic effectiveness.

The preferred embodiment of the invention primarily includes Salvinorin A since the other unextracted moieties, contained within the leaf specks, are mostly inactive and consist of virtually negligible (i.e. less than 50 μg) amounts of Salvinorin “B” through “F” and Divinatorin “A” through “E”. The benefit of ingesting both the active and mostly inactive constituents of Salvia divinorum is to maintain its existing psycho-spiritual and/or medicinal potential by not ridding the formulation of its natural origin. In other embodiments, any amount of one or more of the ingredients Salvinorin “B” through “F” and Divinatorin “A” through “E” may be incorporated into the gum.

The gum base is typically the component with greatest concentration in chewing gum formulations. Since gum is generally known to be a stiff and solid substance it generally does not provide for a soft or “linear” chew characteristic. The jaw-pressure required when chewing is inconsistent and becomes too high after just a few minutes when the gum base is in high concentration. Since it can degrade the texture and make the gum as a whole difficult to chew the preference in this invention is to lower its percent content relative to other constituents following customary embodiments in the art. The gum base's percent content, by weight of the total gum composition, is also chosen and presented in this invention to help regulate the affinity and thus the efficiency in releasing the active ingredients.

When still embedded in the leaf, the active component of S. divinorum is substantially water- and saliva-insoluble so a base emulsifier system has added importance in this invention to improve the lipophilic balance of the active ingredients when incorporated together with the gum base. Similarly, inorganic fillers enhance chew-ability and even enhance the absorption of the active ingredient. As a result of the very low relative content of Salvinorin A in the gum, extended periods of chewing time can be easily reached due to not needing too many other constituents to mask its flavor. Softeners in general as part of this invention are used to further reduce the viscosity of the gum base since hard gum can also damage teeth and gums. Base emulsifier systems also help to emulsify liquids, absorb moisture, and facilitate release of the active ingredients upon mastication.

Fluoride is often added to gum base to additionally reduce tooth decay. Other excipient means such as binders, disintegrants, surfactants, anti-adherents, glidants, lubricants, preservatives, and sorbents can also be added to the gum formulation of the present invention.

Stevia extract is a non-cariogenic sweetener and the sweetener of choice in the preferred embodiment of this present invention. Similarly, Polyvinyl Acetate or its most similar natural equivalent is the elastomer resin gum-like additive of choice due it is hydrophilic characteristics, low weight, and ability to provide for a softer, less brittle, and less sticky gum base while also providing a fast, initial release of the Salvinorin A active ingredient.

Another embodiment of the invention is to use a gum base comprised of one or combinations of natural elastomer resins, synthetic elastomer resins, softeners (e.g. emulsifiers and/or plasticizers), and elastomer solvents. Other excipient means comprise part of this invention, consisting of diluent(s) as viscosity modifiers, humectant(s) to remove moisture, abrasive(s) to smooth or make the surface of the gum rough, and antioxidant(s) to protect and stabilize the gum base. Other resins, fat(s), oil(s), and wax(es) are considered part of the softening agents and fillers, which are also considered part of the insoluble gum base, though various compounds can affect the release rate of active ingredients. Elastomer solvents are considered rosin-resin compounds, elastomers are ester plasticizers, and softeners are also known as plasticizers for their shaping and forming ability of the material to which they are added. Emulsifiers generally balance out the consistency in a liquid suspension of any incompatible constituents of the formulation presented herein before or after commencement of mastication. The buffering agents are added to set the saliva pH to within +/−2.5 of optimal and efficient absorption of Salvinorin A.

Another embodiment of the invention is to use entirely organic (i.e. non-synthetic) components such that the entire gum base can be swallowed. Since it is all natural it can be swallowed as a fiber supplement. Swallowing avoids the inconvenience of finding a trash receptacle each time one expels a gum at the end of the experience or prematurely per desire. This nuanced characteristic of the gum formulation in this invention further avoids the social problem of used gum being inconsiderately placed on non-trash objects. Typically, swallowed gum will very quickly cease providing any appreciable release rate of active ingredient sufficient to maintain the design concentration.

Another embodiment of the invention is to use natural elastomer resins comprised of one, or combinations of: Chicle, Jelutong, Sorva, Gutta Percha, Gutta Hang Kang, Crown Gum, Lechi Di Caspi, Soh, Siak, Katiau, Balata, Pendare, Perillo, Sorva, Massaranduba Balata, Massaranduba Chocolate, Nispero, Rosindinha, Malaya, Crepe Caoutchouc, Smoked Caoutchouc Latex, Liquid Caoutchouc Latex, Guayule, Dammar resin, Mastix resin, Malsa compound PU-C, Picolyte resin, and Chiquibil gum. Another embodiment of the invention is to use synthetic elastomer resins comprised of one, or combinations of: Polyisobutylene Copolymer, Isobutylene-isoprene Copolymer, Polyvinyl Ester, Polyvinyl Acetate, Polyethelene, Polyisoprene, Polyethylene, Vinyl Acetate, Vinyl Laurate, Vinyl Laurate, “Dreyco”, “Paloja”, “Firm Paloja”, and “Berguna”. Another embodiment of the invention is to use softeners comprised of one, or combinations of: Lanolin, Sodium Stearate, Potassium Stearate, Glycerol Triacetate, Glycerol Monostearate, Glycerine, Vaseline, Propylene glycol, Hexylene glycol, Tallow, Hydrogenated Tallow, Cocoa Butter, Fully hydrogenated veg. oil, Partially hydrogenated veg. oil, Egg yolk, Polysorbate, and Sorbitan. More preferably, the embodiment of the invention is to use softeners comprised of one, or combinations of: Monoglyceride, Diglyceride, Triglyceride, Acetylated Monoglyceride, Stearic fatty acid, Palmitic fatty acid, Oleic fatty acid, and Linoleic fatty acid as the softeners.

Another embodiment of the invention is to use elastomer solvents comprised of one, or combinations of: Methyl, Partially hydrogenated natural rosin ester, Natural glycerol ester of polymerized rosin, Natural partially dimerized rosin ester, Pentaerythritol esters of partially hydrogenated rosin, Pentaerythritol esters of fully hydrogenated rosin, Partially hydrogenated methyl ester of rosin, Fully hydrogenated methyl ester of rosin, and Synthetic Terpene resin.

Another embodiment of the invention is to use fats, oils, and waxes comprised of one, or combinations of: Monoglyceride, Diglyceride, Canola Oil, Soybean Oil, Cottonseed Oil, Mineral Oil, Olive Oil, Hydrogenated Coconut Oil, Beeswax, Microcrystalline Wax, Other natural Wax, Polyethylene Wax, Paraffin Wax, Fischer-Tropsch Wax, Rice bran Wax, Carnauba Wax, and Candelilla Wax. Such components, as well as emulsifiers, may be premixed with the active ingredient prior to incorporation into the gum, to allow such close association needed so that these additives can affect the release rate of active ingredient during mastication.

Another embodiment of the invention is to use fillers comprised of one, or combinations of: Calcium Carbonate, Magnesium silicate, Dicalcium phosphate, Misc. Preservative, Calcium carbonate, Magnesium carbonate, Talc, Sodium Sulphate, Aluminum Oxide, Kaolin, Silicum Oxide, and Calcium Phosphate, Metallic Alumina mineral salt, Metallic Aluminum Hydroxide mineral salt, Metallic Aluminum silicate mineral salt, Ground limestone, Magnesium silicate, Aluminum silicate, Clay, Titanium oxide, Monocalcium phosphate, Dicalcium phosphate, Tricalcium phosphate, and Cellulose polymers.

Another embodiment of the invention is to use sweeteners comprised of one, or combinations of: Stevia rebaudiana, Sucrose, Glucose, Dextrose, Fructose, Saccharin, Cyclamic acid, Sucralose, Dihydrochalcone, Glycyrrhin, Sorbitol, Mannitol, Xylitol, Hexa-resorcinol, Glycine, Aspartame, Cyclohexyl sulfamate, Acesulfame-k, Glycyrrhizinate, Hydrogenated starch hydrolysates, Maltitol, Richardella dulcifica, Diascoreophyllum cumminsii, Alitame, and Neotame.

Another embodiment of the invention is to use flavorants comprised of one, or combinations of: Peppermint, Spearmint, Wintergreen, Cinnamon, Menthol, Essential oils, Citrus oils, Fruit essences, Clove oil, and Anise.

Another embodiment of the invention is to use encapsulating coating material comprised of one, or combinations of: Oleaginous fat, Oleaginous oil, Saccharides, Proteins, Non-toxic polymers, and Stearine. Such components may be premixed with a portion of the active ingredient prior to incorporation into the gum, to allow such close association needed so that these additives can affect the release rate of active ingredient during mastication.

Another embodiment of the invention is to use antioxidants comprised of one, or combinations of: Carnosol, Rosmanol, and Rosmaridiphenol.

Another embodiment of the invention is to use basic (i.e. alkaline) buffer chemicals comprised of one, or combinations of: Alkali carbonates, Potassium based, Sodium based, Calcium based, Magnesium based, Aluminum salts based, and Ammonium hydroxide. Another embodiment of the invention is to use acidic buffer chemicals comprised of one, or combinations of: Citric, Malic, Fumaric, Succinic, Tartaric, Formic, Acetic, Propanoic, Butyric, Valeric, Oxalic, Malonic, Glutaric, Adipic, Glycolic, Aspartic, Pimelic, Maleic, Phthalic, Isophthalic, Terphthalic, Glutamic, Lactic, Hydroxyl acrylic, Alpha hydroxyl butyric, Glyceric, Tartronic, Salicylic, Gallic, Mandelic, Tropic, Ascorbic, Gluconic, Cinnamic, Benzoic, Phenylacetic, Nicotinic, Kainic, Sorbic, Pyrrolidone, Carboxylic, Trimellitic, Benzene sulfonic, Toluene sulfonic, Potassium dihydrogen phosphate, Sodium hydrogen sulfite, Sodium dihydrogen phosphate, Potassium hydrogen sulfite, Sodium hydrogen pyrosulfite, Sodium hexametaphosphate, Sodium pyrophosphate, Potassium pyrophosphate, Sulfamic, Ortho-phosphoric, Pyro-phosphoric, Propionic, Tannic, and Sodium carbonate.

Whitening additives can also be used comprised of one, or combinations of: Kaolin, Calcium carbonate, Silicon dioxide, Titanium dioxide, and Cellulosic material. Fluoride additives can also be used comprised of one, or combinations of Alkali metal, Ammonium, Stannous, Stannous chlorofluoride, Potassium stannous, Alkali metal monofluorophosphates, and Ammonium monofluorophosphate.

The total gum mass in a single piece in the preferred embodiment of this invention is in the range of 0.5 to 4 g, but preferably with a mass of 1 to 3 g.

The Salvinorin A active ingredient in the preferred embodiment is incorporated in the range of 0.01 to 1.4 mg and thus 0.00025% to 0.28% by percentage of the total gum mass. In alternative embodiments, the Salvinorin A active ingredient is incorporated into the gum in the range of 0.01 to 0.1 mg, or alternatively of 0.1 to 0.5 mg.

The water insoluble gum base in the preferred embodiment is in the range of 15% to 75% but preferably in the range of 20% to 45% by weight of the total. The hydrophilic sweetener(s) in the preferred embodiment is in the range of 25% to 80% but preferably 35% to 65% by weight of the total. The water soluble buffer chemical(s) in the preferred embodiment is in the range of 0% to 10% but preferably 2% to 7% by weight of the total. The natural elastomer(s) in the preferred embodiment is in the range of 20% to 65% but preferably 25% to 55% by weight of the total. The synthetic elastomer(s) in the preferred embodiment is in the range of 0-30% but preferably 1% to 20% by weight of the total. The softener(s)in the preferred embodiment is in the range of 2% to 30% but preferably 10% to 20% by weight of the total. The elastomer plasticizer(s) in the preferred embodiment is in the range of 10% to 60% but preferably 20% to 45% by weight of the total. The filler(s) in the preferred embodiment is in the range of 5% to 50% but preferably 15% to 35% by weight of the total.

The encapsulating material(s) in the preferred embodiment is in the range of 0-15% but preferably 0.5% to 5% by weight of the total. The flavoring agent(s) in the preferred embodiment is in the range of 0.5% to 8% but preferably 1% to 5% by weight of the total. Other excipients in the preferred embodiment are in the range of 2% to 20% but preferably 5% to 15% by weight of the total. The preservative(s) in the preferred embodiment is used in quantities less than 1% by weight of the total.

As part of the present invention, extraction of the active moieties from within the leaves of S. divinorum and purification means are performed in the preferred embodiment with non-polar solvents such as acetone and polar solvents such as naphtha, respectively. An extraction means is necessary since Salvinorin A is naturally bound in the resins. The subsequent purification means of the Salvinorin A constituent may be performed to remove waxy particulates through several washes using a polar solvent, which Salvinorin A is not soluble in. When considering that this invention presents a chewing gum formulation of varying potencies it is pertinent to note that the purification step is only necessary for the highest of the three dosage levels, i.e. the “15x” level discussed herein.

In the preferred embodiment, the extraction means begins with the pinching of the leaves from aged leaves, where “aged” is defined as leaves that have been at a mature size of at least 2 inches in length in the range of 10 to 1000 days, more preferably between 30 and 300 days. Next the leaves are lyophilized by a lyophilizing means, then taken into a dry and dimly lit manufacturing facility since moisture, light, and radiation diminish the potency of the leaves and Salvinorin A. The leaves are then added into a container containing a non-polar solvent consisting of one, or combinations of: Acetone, Ethyl Alcohol (Ethanol), Methyl Alcohol (Methanol), 99% Isopropyl Alcohol (IPA), Dimethylsulfoxide (DMSO), Methylene Chloride, Chloroform, Polyethylene glycol, Polypropylene glycol, 2-propanol, Isopropanol, Glycerin, Mineral oil, Diethylether, Carbontetrachloride, Acetonitrile, Cyclohexane, Acetic acid, Nitromethane, Dioxane, Hexane, Pentane, Acetylene, Pyridineand, Water, and Super Critical Fluids (SCFs). SCFs are mentioned by reference only and do not comprise part of this invention without a licensing agreement. Acetone is used herein as the solvent of choice since Salvinorin A is highly soluble in it.

A preferred embodiment of this invention presents the use of a Soxhlet apparatus for the extraction process, which retains the solid material in a holder and allows the solvent to flow through the material. The solvent is heated to a range of temperatures between 50° F. and a degree below the boiling temperature of the solvent used, most preferably between 100 and 150° F. The solvent is then added to just cover the powdered leaf, within an inch from the surface. The solvent and powdered S. divinorum leaves are then soaked, mixed, steeped, and/or stirred for between 3 and 60 minutes, in the preferred embodiment, this being preferably between 5 and 15 minutes.

After the leaf particulates (e.g. tannins) have settled, upon completing the stirring, which takes place for between 10 and 200 minutes, the solution is poured into a second container. As the solution is poured off the powdered leaves, the poured off solution pulls with it a large portion of the Salvinorin A. More solvent is then poured into the original container so as to most efficiently extract, in total, greater than 90% of the available active ingredient in the leaves. The stirring and pouring process is repeated between one and ten more times, but preferably three to six more times, whereupon on each occasion the solution is combined with the contents in the second, separate container. The process of pouring out the solution from the settled particulates into a third container without adding new solvent is repeated one to three more times, preferably twice in the preferred embodiment.

As a major part of the preferred embodiment of the present invention, a non-cariogenic sweetener is dissolved with the purified or non-purified Salvinorin A in the final, resulting solution. The addition of a sweetener to the blend eliminates the bitter taste aspect. Preferably the sweetener is non-nutritive and in the preferred embodiment is an extract of Stevia. The ratio of the sweetener to S. divinorum and Salvinorin A is not explicitly provided herein but instead is determined implicitly as part of their respective concentrations relative to the total weight of the gum. This ratio is chosen such that sufficient masking of the taste of the botanical and its extract is achieved while it is also sweet enough for overall organoleptic pleasure when combined with the chewing gum base.

The remaining solvent is then vacuum processed to completely remove it and thus speed up the evaporation process without relying entirely on air facilitated evaporation. The extracted Salvinorin A is then purified through one to five washes with a polar solvent. The resulting product is a white crystalline form of Salvinorin A accompanied by dark, waxy chlorophyll compounds, which aid in the sublingual and buccal absorption. Naphtha is used in this step as the solvent of choice since Salvinorin A is highly soluble with it. Another effective polar solvent is xylene. Salvinorin A can also be purified in another embodiment of this invention by silica gel flash column chromatography.

The polar solvent is then vacuumed off the extract or allowed to evaporate naturally in open air. The denatured components of each of these solvents are thus evaporated off prior to inclusion with the excipients and the other constituents of the gum formulation. The end result is a dark green substance where the dark color is due to the components that originated from the leaf cuticle. The coagulated compounds are then immediately ground and crushed into a powder form by the use of a mill means. The milling procedure includes crushing, cutting, grinding, and chopping, before mixing with the excipients.

The extract is further dried by a slight application of heat no hotter than 150° F. then scraped off and re-milled. The dry processing, including blending with the excipients and other gum constituents, is performed as part of this invention to protect the sensitive agent from moisture-caused degradation and to prevent overall destabilization of the active moieties. A preferred embodiment of this latter blending step in the process is performed through direct compression. The subsequent even and consistent dispersion of the resultant active powder throughout the entire gum base is a preferred embodiment of this manufacturing process.

As a major part of the present invention, the standard dosing scheme of three different concentration levels of Salvinorin A, including the leaf specks, are precisely formulated to induce predetermined levels of effect over a predetermined range of time. Concentration levels are denoted with a number prefixing a letter “x” and in this invention are presented exclusively as “1x”, “5x”, and “15x”, where the number N is approximately the number of times the concentration of Salvinorin A in the chewing gum is multiplied by the concentration in an unextracted leaf. A 1x dose might be a dose found in a single leaf, which is not necesasarily the dosage obtainable from chewing a leaf. In this invention the number N is chosen to specifically map to three of the standardized levels outlined in Daniel Siebert's six-pointed S-A-L-V-I-A experiential rating scale (Siebert 2009). This objective quantification of Salvinorin A is performed akin but ultimately unlike the Recommended Daily Value (RDV) that is measured relative to an average human body type by the Food and Drug Administration.

In order to achieve an “S” level for this scale, as part of this chewing gum formulation, 0.01 to 0.2 mg, for example 0.1 to 0.2 mg, of Salvinorin A powder is mixed into the single serving of gum base. Similarly, to achieve an “A” level 0.2 to 0.8 mg of Salvinorin A powder is mixed into the gum base and to achieve an “L” level 0.8 to 1.4 mg of Salvinorin A powder is mixed into the gum base. Concentration levels above 1.4 mg would not typically be expected to be used in a final salable product. The actual concentration levels will be above each of these values by trivial amounts ranging from 1 to 50 μg due to the saliva extraction means from the leaf specks. Overall, this dosing regimen equates to approximately 0.15 to 15 μg per kg of body weight. By comparison, it is noted that lab tests with mice are typically performed in higher concentrations ranging from 125 to 2,000 μg per kg but the doses presented herein are not unreasonably low due to the 5x to 10x higher metabolism rate in rodents.

In the “1x” category, which maps to the psychotropic “S” level, it is expected that the gum will contain negligible amounts of pre-extracted constituents of S. divinorum and thus will contain pharmacological quantities of the active compound Salvinorin A so low that such amounts may be tantamount to a “placebo gum” for some individuals. The “5x” extract fortified gum, which maps to the psychotropic “A” level, will contain more Salvinorin A. Similarly, the “15x” extract fortified gum, which maps to the psychotropic “L” level, will contain even greater concentrations of Salvinorin A by weight of the total volume of the gum.

The resultant effect from chewing a gum embodiment presented herein is dependent on the body weight, age, genetic makeup, sex, and diet. Irrespective of the concentration level, the recommended serving size, as presented in the preferred embodiment, will be comprised of no more than one S. divinorum stick of gum per day and no more than five sticks per week. An overdose can occur if the number of pieces of gum being chewed consecutively in a short time frame is greater than this recommended regimen but the risk this poses is behavioral-related and not biological due to the ultra low toxicity of Salvinorin A. This inventor believes out of his extensive research that overdose should therefore be characterized in terms of one's subjective preference and tolerance.

As an embodiment of the present invention, the consistent time-release of Salvinorin A is presented, including a technique to match the time release and subsequent subsiding effect of Salvinorin A with the inter-granulated sweetener. In this fashion when the flavor has dissipated away the individual will know that the Salvinorin A is likewise no longer active.

The simple release of Salvinorin A is not sufficient, in the preferred embodiment, for facilitating psycho-spiritual progress. The subsequent efficient absorption of some or all of it into the body, i.e., the bloodstream, is therefore of greater importance. The technique to accomplish the necessary time release is presented herein with the use of a buffer chemical means, including the characterization of the percent absorbed over a certain period of time. The buffer chemicals can be used for the quick “bonus” release and the naturally occurring saliva amylases (i.e. enzymes) in the oral cavity facilitate the reuptake of the less soluble active moieties that have been recaptured in the gum base once the buffer chemicals have been depleted. This technique serves to provide a consistent pharmacological effect to the individual for the duration of the chewing experience, which normally lasts between 30 and 60 minutes.

In one embodiment the gum contains both alkaline buffers, such as carbonates, e.g., alkali or alkaline earth carbonates, which promote fast absorption of the active ingredient by mucus membranes, and also includes agents such as bemusifiers, which may be admixed with hydrophobic materials, to hold active ingredient and provide an extended release of active ingredient such that the level of active ingredient in the user maintains at a stable rate. As used herein, active ingredient is understood to be Salvinorin A that is at least partially separated from residual Salvia divinorum leaf residue remaining after extraction.

Buffer chemicals are primarily active during the first stage. They enhance absorption in the oral mucosa of the free-unionized active ingredient since the buffer chemicals alter the pH of saliva. Alteration of the pH in the oral cavity is necessary for optimal absorption of Salvinorin A. Repeated chewing in the latter stage allows for the saliva to further extract the milled Salvinorin A crystals from the gum base and also to release the unextracted active agents from the leaf specks. This latter stage also prolongs the pharmacological effect due to the sustaining elevated pH levels. This phenomenon is caused by the continued action of chewing and more specifically from a continued high quantity of saliva, which naturally causes the release of carbonate buffers to replace the expended synthetic buffers. Combined, both of these stages serve to facilitate the secretion of Salvinorin A before deactivation in the stomach.

This two staged approach, which provides for quick release and prolonged blood-level concentrations of Salvinorin A, is often referred to as multi-phasic release in the art of chewing gum formulations. The preferred embodiment of this invention presents a formulation where 25% to 50% absorption of Salvinorin A occurs within the first 15 minutes and another 15% to 45% is released in the subsequent 45 minutes. Of course, with a product that is intended for shorter time periods such as one half an hour, the formulation may release 25% to 50% of active ingredient in the first ten minutes or so, and then release 50% to 75% of the product over the extended release portion of time, say the subsequent 20 minutes. Any higher release rates in the first stage are not likely to be as useful due to the wasteful and inefficient transmucosal absorption that takes place inherent in the act of swallowing when chewing a gum. Other undesirable and unintended effects such as gastrointestinal stress and organoleptic displeasure, which is common to other chewing gums that contain actives such as nicotine, can be avoided with limited release in the first 15 minutes.

The preferred embodiment of this invention comprises the technique of slowly incrementing the dosage by suggesting that individuals first introduce themselves to the “1x” version prior to partaking of the higher levels. For inexperienced individuals the time release can be aborted by expelling the gum from the oral cavity of the mouth if the intensity of the effect is beyond the desired level. Other than this sudden cessation of chewing to prevent dysphoria, the preferred embodiment of this invention recommends that an individual chew slowly at first to modulate the effects; the nominal chewing rate in the preferred embodiment is one chew every 3 to 6 seconds. Employing the “chew and park” technique as opposed to regular, consistent chewing is also effective at modulating the effects.

For facilitating quicker intake, experienced users can minimize swallowing the juices since absorption primarily occurs transmucosally (i.e. buccally, sublingually, and pharyngeally). The individual can also brush their teeth before chewing the gum to enhance the absorption. Another technique is simply to continue chewing for an entire hour at which point the large majority of Salvinorin A is released. Both of these decelerated and accelerated ingestion techniques are preferred embodiments of the present invention.

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed novel system, structure, or manner.

What is claimed is:

1. A chewing gum for enhancing psycho-spirituality, comprising:
(a) a gum base;
(b) extracted Salvinorin A; wherein the formulation is adapted to provide 25% to 50% release of Salvinorin A within the first 15 minutes of chewing and an additional 15% to 45% released in the subsequent 45 minutes of chewing.

2. The chewing gum of claim 1 wherein the gum comprises 0.01 to 1.4 mg of Salvinorin A.

3. The chewing gum of claim 1 wherein the gum comprises 0.2 to 0.8 mg of Salvinorin A.

4. The chewing gum of claim 1 wherein the gum comprises 0.8 to 1.4 mg of Salvinorin A.

5. The chewing gum of claim 1 wherein the gum comprises an alkaline buffer.

6. The chewing gum of claim 5 wherein the buffer is in the range of 2% to 7% by weight.

7. The chewing gum of claim 1 further comprising a base emulsifier and a non-cariogenic sweetener.

8. The chewing gum of claim 1 further comprising unextracted leaf fragments from Salvia divinorum.

9. The chewing gum of claim 1, wherein the chewing gum is adapted to provide a Salvinorin A time-release profile on chewing to provide a consistent pharmacological effect for a period of time between 30 and 60 minutes, wherein the pharmacological effect is the “A” level on the six-pointed S-A-L-V-I-A experiential rating scale.

10. The chewing gum of claim 1, wherein the chewing gum is adapted to provide a Salvinorin A time-release profile on chewing to provide a consistent pharmacological effect for a period of time between 30 and 60 minutes, wherein the pharmacological effect is the “L” level on the six-pointed S-A-L-V-I-A experiential rating scale.

11. The chewing gum of claim 1 further comprising a base emulsifier.

12. The chewing gum of claim 1 further comprising encapsulating material.

13. The chewing gum of claim 1 further comprising waxy chlorophyll compounds extracted from Salvia divinorum.

14. A chewing gum formulation comprising
(a) a gum base;
(b) extracted Salvinorin A; wherein the formulation is adapted to provide a Salvinorin A time-release profile to provide on chewing a consistent pharmacological effect for a period of time between 30 and 60 minutes, wherein the pharmacological effect is the “A” level or the “L” level on the six-pointed S-A-L-V-I-A experiential rating scale.

15. The gum of claim 14 further comprising an alkaline buffer.

16. A method for enhancing psycho-spirituality comprising the step of chewing the gum of claim 1 and tranmucosally absorbing the ingredients which are released by chewing into the body.

17. The method of claim 16 wherein chewing the gum will induce a predetermined level of effect over a predetermined range of time between 30 and 60 minutes.

18. The method of claim 17 wherein the predetermined level is the “A” level on the six-pointed S-A-L-V-I-A experiential rating scale.

19. The method of claim 17 wherein the predetermined level is the “L” level on the six-pointed S-A-L-V-I-A experiential rating scale.
